(19) United States
(12) Patent Application Publication
(10) Pub. No.: US 2024/0407726 A1
(43) Pub. Date: Dec. 12, 2024

(54) PATIENT MONITOR PLACEMENT INDICATOR

(71) Applicant: Masimo Corporation, Irvine, CA (US)

(72) Inventors: Jeroen Poeze, Rancho Santa Margarita, CA (US); Marcelo M. Lamego, Coto De Caza, CA (US)

(21) Appl. No.: 18/750,508

(22) Filed: Jun. 21, 2024

Related U.S. Application Data
(63) Continuation of application No. 18/048,416, filed on Oct. 20, 2022, now Pat. No. 12,042,300.
Application No. 18/048,416 is a continuation of application No. 16/780,762, filed on Feb. 3, 2020, now Pat. No. 11,504,062.
Application No. 16/780,762 is a continuation of application No. 15/910,542, filed on Mar. 2, 2018, now Pat. No. 10,575,779.
Application No. 15/910,542 is a continuation of application No. 14/209,844, filed on Mar. 13, 2014, now Pat. No. 9,936,917.
(60) Provisional application No. 61/785,180, filed on Mar. 14, 2013.

(51) Int. Cl. A61B 5/00; A61B 5/1455

(52) U.S. Cl. CPC A61B 5/684; A61B 5/14551; A61B 5/6826; A61B 5/742; A61B 5/7405; A61B 5/743

(57) ABSTRACT

A monitoring device for measuring one or more physiological parameters of a medical patient can include a finger clip sensor connected to a monitor. A placement indicator helps the patient to properly position the sensor. The monitor can display a message alerting the patient to reposition the sensor. The device can delay measurement until the sensor is properly positioned.

### RELATED APPLICATIONS

### Incorporation by Reference to any Priority Applications

Any and all applications for which a foreign or domestic priority claim is identified in the Application Data Sheet as filed with the present application are incorporated by reference under 37 CFR 1.57 and made a part of this specification.

### BACKGROUND

### Field of the Disclosure

The disclosure herein relates generally to patient monitoring with non-invasive physiological sensors. More specifically, the present disclosure relates to devices and methods for aiding a patient in positioning the physiological sensor for more accurate results.

### Description of the Related Art

The standard of care in caregiver environments includes patient monitoring through spectroscopic analysis using, for example, a pulse oximeter. Devices capable of spectroscopic analysis generally include a light source(s) transmitting optical radiation into or reflecting off a measurement site, such as, body tissue carrying pulsing blood. After attenuation by tissue and fluids of the measurement site, a photodetection device(s) detects the attenuated light and outputs a detector signal(s) responsive to the detected attenuated light. A signal processing device(s) process the detector(s) signal(s) and outputs a measurement indicative of a blood constituent of interest, such as glucose, oxygen, met hemoglobin, total hemoglobin, other physiological parameters, or other data or combinations of data useful in determining a state or trend of wellness of a patient.

In noninvasive devices and methods, a sensor is often adapted to position a finger proximate the light source and light detector. For example, noninvasive sensors often include a clothespin-shaped housing that includes a contoured bed conforming generally to the shape of a finger. The contoured bed can help position and stabilize the finger for measurement.

### SUMMARY

This disclosure describes embodiments of noninvasive methods, devices and systems for measuring physiologically relevant patient characteristics and for determining proper positioning of the physiological sensor for a specific patient. The monitoring device can determine a reference position of the sensor with respect to the patient when the sensor is properly positioned on the patient. After the reference position is determined, the monitoring device can use the reference position to help the patient maintain the position of the sensor in the appropriate position for clear and accurate measurements. In certain embodiments, when the device detects improper positioning, a display on a monitor alerts or provides an indication to the patient that the sensor is out of position. In some embodiments, the monitoring device can delay taking measurements until the sensor is returned to the reference position. In some embodiments, the display on a monitor associated with the sensor displays an interactive game for the patient to play, which can help the patient position the monitoring device.

For purposes of summarizing the disclosure, certain aspects, advantages and novel features have been described herein. It is to be understood that not necessarily all such advantages can be achieved in accordance with any particular embodiment disclosed herein. Thus, the disclosure described herein can be embodied or carried out in a manner that achieves or optimizes one advantage or a group of advantages as taught herein without necessarily achieving other advantages as can be taught or suggested herein.

### BRIEF DESCRIPTION OF THE DRAWINGS

Throughout the drawings, reference numbers can be re-used to indicate correspondence between referenced elements. The drawings are provided to illustrate embodiments described herein and not to limit the scope thereof.

FIG. 1 illustrates an exemplary handheld monitor and an exemplary noninvasive optical sensor of a patient monitoring system, according to embodiments of the disclosure.

FIG. 2 illustrates a monitoring device in accordance with one embodiment with the sensor attached to a patient's finger.

FIG. 3 illustrates a block diagram illustrating data processing of signals used for placement indication.

FIG. 4 illustrates an embodiment of a flowchart for operation of the monitoring system utilizing sensor placement indication

FIG. 5 illustrates an exemplary embodiment of an interactive game displayed on a monitor associated with a physiological sensor according to embodiments of the disclosure.

### DETAILED DESCRIPTION OF PREFERRED EMBODIMENTS

A monitoring device in accordance with one embodiment of the present disclosure comprises a physiological sensor including a detector housing for attachment to a measurement site. The measurement site can be, for example, a patient's finger, and the detector housing can comprise a clothespin-shaped housing that includes a contoured bed conforming generally to the shape of a finger. The monitoring device further comprises one or more monitors that process and/or display the sensor's output and/or other information. The monitors can include various components, such as a sensor front end, a signal processor, a display, etc.

The sensor can be integrated with a monitor, for example, into a handheld unit including the sensor, a display and user controls. In other embodiments, the physiological sensor can be connected to and communicate with the monitor(s) via a cable or a wireless connection. The communication can be via wire(s), cable(s), flex circuit(s), wireless technologies, or other suitable analog or digital communication methodologies and devices to perform those methodologies. Many arrangements allow the sensor to be attached to the measurement site while the monitor is attached elsewhere on a patient, such as the patient's arm, placed at a location near the patient, such as a bed, shelf or table, or held by the patient.

Reference will now be made to the Figures to discuss embodiments of the present disclosure.

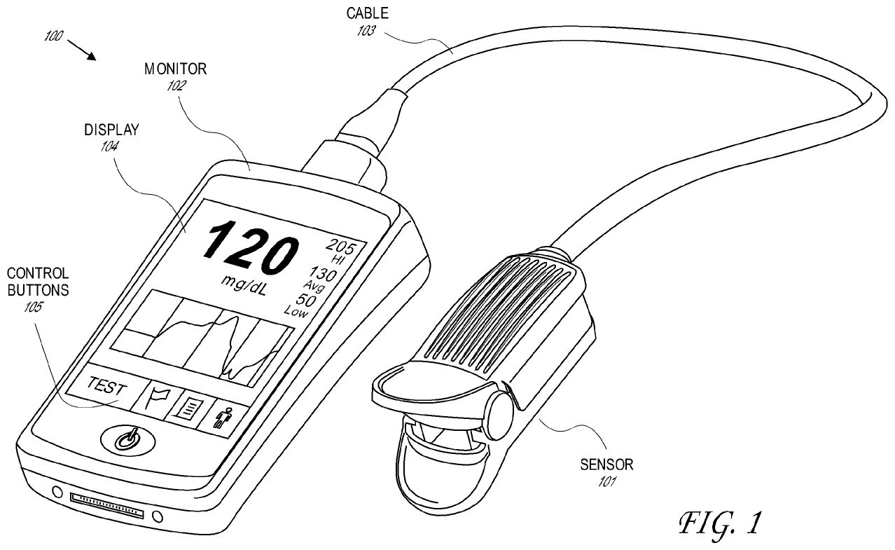

FIG. 1 illustrates one example of a monitoring device 100. In the depicted embodiment, the monitoring device 100 includes a finger clip sensor 101 connected to a monitor 102 via a cable 103. In the embodiment shown, the monitor 102 includes a display 104, control buttons 105 and a power button. Moreover, the monitor 102 can advantageously include electronic processing, signal processing, and data storage devices capable of receiving signal data from said sensor 101, processing the signal data to determine one or more output measurement values indicative of one or more physiological parameters of a monitored patient, and displaying the measurement values, trends of the measurement values, combinations of measurement values, and the like. The electronic processing, signal processing, and data storage devices of the monitor 102 can also be capable of receiving data, processing the data, generating displays based at least in part on the data as further described below, and directing the system when to record measurements of the physiological parameters of interest.

The cable 103 connecting the sensor 101 and the monitor 102 can be implemented using one or more wires, optical fiber, flex circuits, or the like. In some embodiments, the cable 103 can employ twisted pairs of conductors in order to minimize or reduce cross-talk of data transmitted from the sensor 101 to the monitor 102. Various lengths of the cable 103 can be employed to allow for separation between the sensor 101 and the monitor 102. The cable 103 can be fitted with a connector (male or female) on either end of the cable 103 so that the sensor 101 and the monitor 102 can be connected and disconnected from each other. Alternatively, the sensor 101 and the monitor 102 can be coupled together via a wireless communication link, such as an infrared link, radio frequency channel, or any other wireless communication protocol and channel.

The monitor 102 can be attached to the patient. For example, the monitor 102 can include a belt clip or straps that facilitate attachment to a patient's belt, arm, leg or the like. The monitor 102 can also include a fitting, slot, magnet, LEMO snap-click connector or other connecting mechanism to allow the cable 103 and sensor 101 to be attached to the monitor 102.

The monitor 102 can also include other components, such as a speaker, power button, removable storage or memory (e.g., a flash card slot), an AC power port, and one or more network interfaces, such as a universal serial bus interface or an Ethernet port. For example, the monitor 102 can include a display 104 that can indicate measurements obtained by the physiological sensor, prompt a user for input, or display messages and/or games as discussed below.

In addition, although a single sensor 101 with a single monitor 102 is shown, different combinations of sensors and device pairings can be implemented. For example, multiple sensors can be provided for a plurality of differing patient types or measurement sites or even patient fingers.

Example monitoring devices are described in U.S. Publication No. 2010/0030040, filed Aug. 3, 2009, titled “Multi-Stream Data Collection System for Noninvasive Measurement of Blood Constituents” and U.S. Publication No. 2010/0010326, filed Jul. 2, 2009, titled “Contoured Protrusion for Improving Spectroscopic Measurement of Blood Constituents,” the disclosures of which are both hereby incorporated by reference in their entirety. An example commercial embodiment of a monitoring device is available from Masimo® Corporation of Irvine, California.

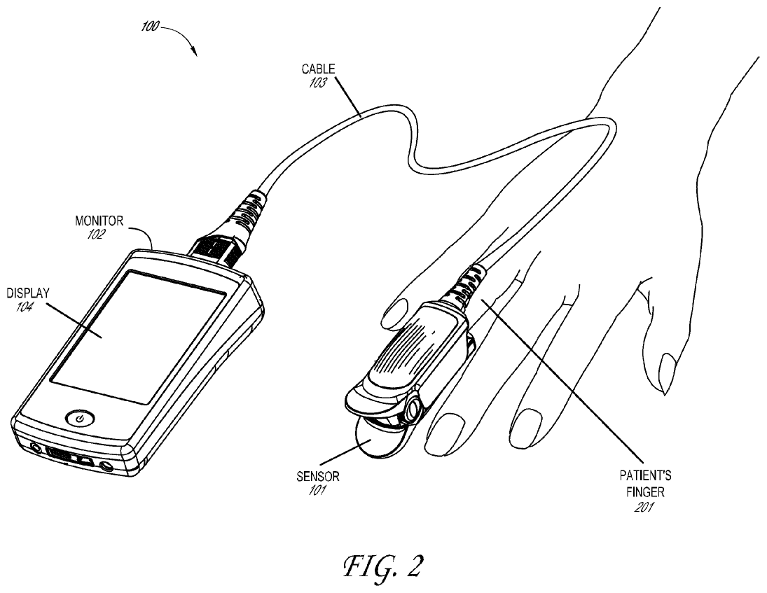

FIG. 2 shows a monitoring device 100 in accordance with one embodiment of the present disclosure with the sensor attached to a patient's finger 201. The patient' finger 201 has a range of movement within the sensor 101. The patient's finger 201 can move in all directions, such as longitudinally, vertically, and from horizontally. When the patient's finger 201 is not correctly positioned with the sensor 101, the sensor 101 can provide erroneous data.

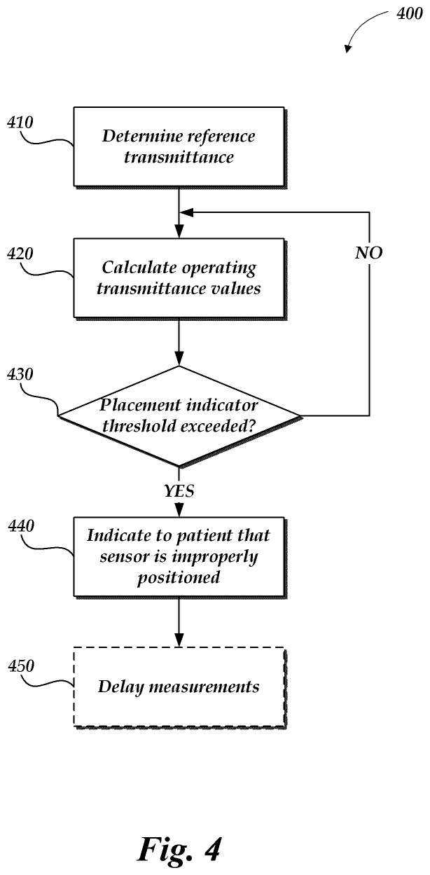

Before attaching the physiological sensor 101 to the patient and beginning monitoring, a user can input information regarding the patient, e.g., whether the patient is an adult or child, into the monitor 102 via, for example, control buttons 105 on the monitor housing or touch screen display buttons 305 (shown in FIG. 4). In some embodiments, the system displays a message on the display 104 prompting the user to input such information regarding the patient or type of display desired.

When the sensor 101 is placed on the patient's finger 201, the monitor 102 determines a reference position for the sensor 101. A patient's finger is used in this illustrative example, however, the principles discussed herein can apply equally to a sensor being positioned on any measurement site of a patient. The reference position is first determined after the sensor 101 has been properly positioned on the patient's finger 201. A user, such as a caregiver, can help correctly position the sensor 101 on the patient's finger. In order to set the reference position the user can provide an indication (e.g., pushing a button) to the monitor 102 to store the current position as the reference position. The monitor 102 determines the reference position by calculating at least one reference transmittance value based on the positioning of the physiological sensor 101. The reference transmittance values can be stored in nonvolatile storage within the monitor 102. In some embodiments, multiple sets of reference transmittance values can be stored.

The reference position stored by the monitor 102 is subsequently used by the monitor 102 to determine whether the patient's finger 201 is properly positioned within the sensor 101. The monitor 102 can continuously monitor the position of the patient's finger 201 to determine whether it is correctly positioned within the sensor. In some embodiments, when the monitor 102 detects that the patient's finger is not properly positioned within the sensor 101 for accurate measurements, the monitor 102 can delay processing measurements and trigger an appropriate display on the display 104 indicating that the sensor 101 needs to be repositioned. In some embodiments, the monitor 102 can detect the direction and the magnitude of the improper positioning of the sensor 101. The display 104 can provide an indication directing the patient how to adjust the sensor 101 for more accurate measurements, for example, directing the patient to move the sensor 101 longitudinally. The system can continue to provide one or more indications until the sensor 101 is properly positioned by the patient. This feature can help alert the patient that the physiological sensor 101 is positioned incorrectly and needs to be readjusted. The monitor can use the reference position to provide instructions to the patient for positioning the physiological sensor without the aid of a medical practitioner. This can help reduce erroneous measurements caused by improper positioning of the physiological sensor.

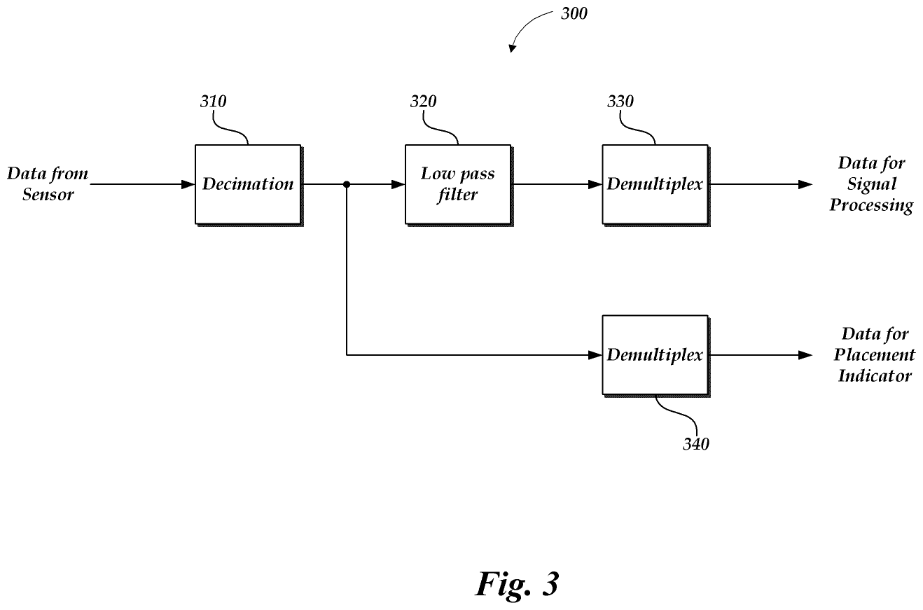

FIG. 3 is a block diagram illustrating a decimation process 300 for signals received from a physiological sensor 101. At block 310, the signal data received from the sensor 101 undergoes a first stage decimation. The first stage decimation can be performed on batches of signal data based on the size of an averaging window. After the first stage decimation at block 310, the signal data can be filtered at block 320 or sent to a demultiplexer at block 340. At block 320, the signal data is filtered using a low pass filter and then sent to a demultiplexer at block 330. The filtered signal data is used by the monitor 102 to perform additional signal processing steps in order to process the physiological signals based on the filtered signal data.

Signal data for the placement indication algorithm is taken after the first stage decimation process and does not undergo filtering through the low pass filter at block 320. The signal data for the placement indicator bypasses the low pass filter 320 and is demultiplexed at block 340. The low pass filter adds additional processing time. By bypassing the low pass filter and using the unfiltered data for the placement indicator, the patient can receive indications that the sensor 101 is not properly positioned more frequently. In some embodiments the patient can receive positioning updates one or more times every second, whereas, filtering the signal data can require one or more seconds of additional processing. The additional time that is required to filter the signal data increases propagation delay and decreases responsiveness of the placement indicator, which can make it difficult for the patient to adjust the sensor in real-time. The signal data does not need to be filtered to calculate placement indication. The signal data that is being used for placement indication does not need to be as high quality as the data that is used to derive the physiological measurements. By using the signal data that has not been processed by the low pass filter reduces propagation delay and improves responsiveness of the placement indicator.

The signal data is used to calculate at least one reference transmittance value in order to determine the reference position. The reference transmittance values are calculated and stored in non-volatile storage. The reference transmittance values in the placement indicator algorithm are compared with the operating transmittance values. The difference between the reference transmittance values and the operating transmittance values, as calculated, can be used as an indicator for placement. The closer the placement indicator is to zero, the closer the finger placement is to the finger placement when the reference transmittance values were acquired. The system can process the operating transmittance values in an open loop, so that the most current value is continuously being recalculated.

A reference transmittance value can be calculated for each LED and channel combination that is being used to determine the placement indication. In some embodiments, all of the LEDs and channels are used to determine placement indication. In some embodiments a subset of the total number of LEDs and channels are used to determine placement indication. In some embodiments, wavelengths that experience high levels of scattering are not used for placement indication.

The value for transmittance for each LED and channel combination is calculated using the following equation:

$$T = \frac{DC}{power \times {PGA}_{g a i n}} \qquad Eq . 1$$

T=transmittance
DC=measured DC intensity
power=estimated LED power based on current and temperature
PGA_{gain}=gain setting of programmable gain amplifier

A transmittance value is calculated using the DC components of the signals. The power is based on the current and the temperature when the measurement is taken. The power compensates for changes in current and temperature in the operating transmittance values that are different from the values of current and temperature used to calculate the reference transmittance values. The monitor can have a transmittance calculator to calculate the transmittance value.

The reference transmittance values and operating transmittance values for each LED and channel combination is used in the following equation to determine a placement indication.

$$P = {\sum }_{i = 1}^{n} \vert \frac{{T}_{i}^{′} - {T}_{i}}{{T}_{i}^{′}} \vert IF {\sum }_{i = 1}^{n} \frac{{T}_{i}^{′} - {T}_{i}}{{T}_{i}^{′}} < 0 , \\ P = {\sum }_{i = 1}^{n} \vert \frac{{T}_{i}^{′} - {T}_{i}}{{T}_{i}^{′}} \vert \qquad Eq . 2$$

otherwise.
Tᵢ=measured transmittance for channel/LED
Tᵢ′=reference transmittance for channel/LED
n=number of LEDs times the number of channels
P=placement indication

Ideally, the calculated value of the placement indication, P, would be zero. As the value of the equation approaches zero, the position of the physiological sensor is closer to the reference position. The value of the placement indicator can be positive or negative. The calculated value of the placement indicator provides a magnitude of difference in positioning between the reference position and the current position of the patient's finger. As the finger is moved closer to the correct positioning, the value of the placement indicator will approach zero. The value of the placement indicator does not provide an absolute unit of measurement. Rather, the value is reduced as the finger approaches the reference position and increased as the finger moves away from the reference position. In some embodiments, the sensor can extrapolate direction based on the positive and negative values associated with the placement indicator value. As such, the system may be able to provide a visual indication to the patient of the approximate direction that the finger needs to be moved within the sensor to substantially align the position of the finger with the reference position. The current position of the finger can be different that the reference position in any direction (e.g., horizontally, vertically, longitudinally, etc.). The monitor can have a placement indication calculator to calculate the placement indication.

Generally, the value of the placement indication will not be zero. Rather than have the patient constantly adjusting the position of the sensor, the monitoring system can have a placement indication threshold. The placement indication threshold is used to determine whether the finger is within an acceptable measurement tolerance. When the placement indication threshold is exceeded then the monitoring system can indicate to the patient that the sensor is not properly positioned.

FIG. 4 illustrates a flowchart for operation of the monitoring system utilizing sensor placement indication 400. At block 410 reference transmittance values are determined. In some embodiments, the reference transmittance values can be determined based on all of the available signals from the sensor. In some embodiments, the reference transmittance is based on a subset of signals from the sensor. The reference transmittance value can be stored in the monitoring system.

At block 420, the current operating transmittance values are calculated using the transmittance algorithm. The monitoring system can continuously calculate and update the transmittance values so that the patient has an updated value of the position of the sensor at regular intervals. In some embodiments, the transmittance values can be updated periodically, such as multiple times a second. In some embodiments, the transmittance values can be are updated at less frequent intervals. In some embodiments, the transmittance values are updated more frequently.

At block 430, the placement indicator algorithm is used to determine the placement indicator transmittance value. The placement indicator transmittance value is compared to a threshold value. If the placement indicator transmittance value is greater than the threshold value then the process proceeds to block 440. If the placement indicator transmittance value is less than the threshold value the monitoring system returns to block 420 and continues to calculate the operating transmittance values. The monitoring system can continue to calculate the operating transmittance values for as long as the sensor is in use by the patient.

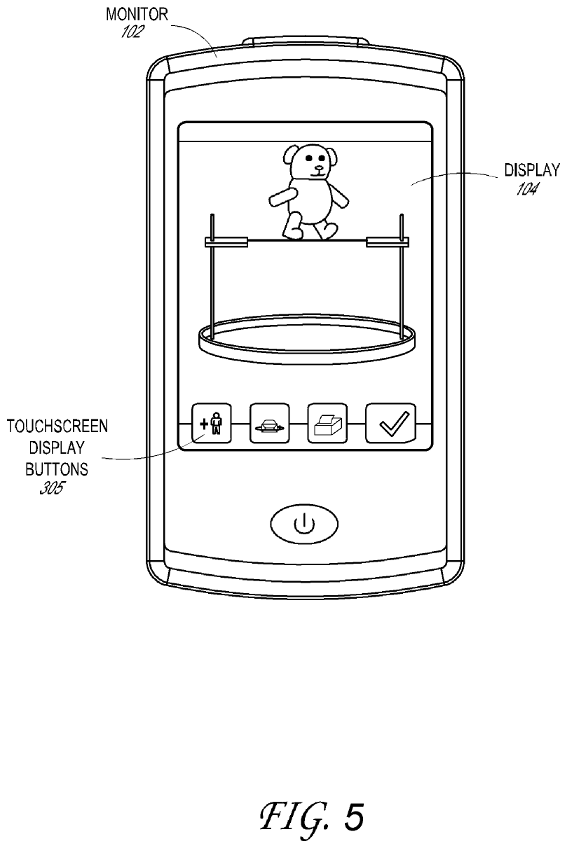

At block 440, the monitoring system can indicate to the patient on a display of the monitor that the sensor is improperly positioned. In some embodiments, the indication to the patient can be an alarm that gets the attention of the patient, such as an audible sound. In some embodiments, the indication can provide a message to the patient that the sensor is improperly positioned. The message can provide text, numeric values and/or other visual cues that can help the patient to reposition the sensor. For example, as the patient repositions the sensor, the display can display arrows that help the patient reposition the patient reposition the sensor. In some embodiments, the indication can be an interactive indicator, such as a game, that is displayed and can help the patient reposition the sensor so that it is correctly positioned on the patient's finger. Games or other interactive displays can be beneficial in instructing pediatric patients that may have difficulty interpreting a textual or other indication. An embodiment of an interactive indicator is further illustrated with reference to FIG. 5.

At block 450, the monitoring system can optionally delay measurements until the placement indicator transmittance value is below the threshold. In some embodiments, the monitoring system may continue to determine measurements even though the placement indicator threshold has been exceeded.

In one embodiment illustrated in FIG. 5, the display 104 displays and initiates an interactive indicator, such as a movement or balance game rather than another type of indication, such as a textual, verbal, or symbolic indicator, in response to the detection of inappropriate positioning. A young child may not be able to read a message directing him or her to adjust the physiological sensor. Even if the child could read such a message, children often have difficulty remaining still even if asked or directed to do so. In certain embodiments, the display 104 is configured to display one or more games such that the patient is motivated by the game to move the physiological sensor to the appropriate position. In addition to helping the patient adjust the monitoring device in order to obtain accurate readings, such games can advantageously provide an activity to occupy and possibly distract the patient.

In one embodiment, the game display comprises images of a teddy bear on a tightrope. Movement of the patient's finger and therefore the sensor corresponds to and causes movement of the bear. The game can provide indications of the direction that the patient needs to move the finger in order to move the bear to the proper position on the tightrope. In other similar embodiments, the bear can be replaced by an animal or human and the tightrope can be replaced by a balance beam, among other things.

One or more indicators, such as interactive indicators (e.g., games) disclosed above, similar interactive indicators, or other indicators can be stored in the monitor's 102 data storage device. Additional games can be loaded to the monitor via a removable storage or memory (e.g., a flash card) or a network interface.

After a user provides input to the monitor 102 indicating that the patient is a child or that a game is to be used rather than a verbal alert message, the display 104 can provide various game options available and prompt the user or patient to select one via a touch screen display or control buttons 105.

While a number of preferred embodiments and variations thereof have been described in detail, other modifications and methods of using and medical applications for the same will be apparent to those of skill in the art. Accordingly, it should be understood that various applications, modifications and substitutions can be made of equivalents without departing from the spirit of the disclosure or the scope of the claims.

1.-20. (canceled)

21. A noninvasive medical monitoring device, the monitoring device comprising:
a physiological sensor comprising a plurality of LEDs and a detector, the plurality of LEDs configured to emit light at one or more wavelengths at a measurement site of a wearer, the detector configured to generate physiological data associated with the one or more light wavelengths after attenuation by tissue at the measurement site of the wearer; and
a transmittance calculator configured to:
determine at least one reference transmittance value based on the physiological data associated with a subset of LED and channel combinations, the at least one reference transmittance value indicative of a reference position of the physiological sensor at the measurement site of the wearer;
determine at least one operating transmittance value based on the physiological data associated with the subset of LED and channel combinations, the at least one operating transmittance value indicative of a current position of the physiological sensor at the measurement site of the wearer;
determine a placement indication value based, at least in part, on the at least on reference transmittance value and the at least one operating transmittance value, the placement indication value indicative of the current position of the physiological sensor at the measurement site relative to the reference position; and
determine whether the placement indication value exceeds a placement indication threshold.

22. The noninvasive medical monitoring device of claim 21, wherein the transmittance calculator is further configured to determine the placement indication value based on a comparison of the at least one reference transmittance value to the at least one operating transmittance value.

23. The noninvasive medical monitoring device of claim 21, wherein the transmittance calculator is further configured to determine the placement indication value based on a determined difference between the at least one reference transmittance value and the at least one operating transmittance value.

24. The noninvasive medical monitoring device of claim 21, wherein the transmittance calculator is further configured to:
responsive to the determination of whether the placement indication value exceeds the placement indication threshold, output an indication that the physiological sensor is out of position.

25. The noninvasive medical monitoring device of claim 24, wherein the indication comprises a visual indication on a display that the physiological sensor is out of position.

26. The noninvasive medical monitoring device of claim 24, wherein the indication comprises an audible alarm, the transmittance calculator further configured to output the alarm to a speaker.

27. The noninvasive medical monitoring device of claim 24, wherein the indication comprises a direction of the current position of the physiological sensor relative to the reference position or a magnitude of a difference between the current position and the reference position.

28. The noninvasive medical monitoring device of claim 21, wherein the transmittance calculator is further configured to delay determination of a physiological measurement associated with the physiological data when the placement indication value exceeds the placement indication threshold.

29. The noninvasive medical monitoring device of claim 21, wherein the monitoring device further comprises a data storage device configured to store the at least one reference transmittance value.

30. The noninvasive medical monitoring device of claim 21, wherein the one or more wavelengths emitted by the subset of LED and channel combinations does not experience high levels of scattering.

31. A noninvasive medical monitoring device, said monitoring device comprising:
a physiological sensor comprising an emitter and a detector, the emitter configured to emit light of one or more wavelengths at a measurement site of a wearer, the detector configured to detect the one or more light wavelengths after attenuation by tissue of the wearer at the measurement site of the wearer, the physiological sensor configured to generate one or more physiological signals associated with the detected light; and
a transmittance calculator configured to:
determine at least one reference transmittance value based, at least in part, on the one or more physiological signals, the at least one reference transmittance value indicative of a reference position of the physiological sensor at the measurement site;
determine at least one operating transmittance value based, at least in part, on the one or more physiological signals, the at least one operating transmittance value indicative of a current position of the physiological sensor at the measurement site;
determine a placement indication value based, at least in part, on the at least one reference transmittance value and the at least one operating transmittance value, the placement indication value indicative of the current position of the physiological sensor at the measurement site relative to the reference position;
generate instructions for the wearer to move the physiological sensor to the reference position when the placement indication value exceeds a placement indication threshold;
responsive to the wearer moving the physiological sensor, update the at least one operating transmittance value; and
update the instructions until the placement indication value does not exceed the placement indication threshold.

32. The noninvasive medical monitoring device of claim 31, wherein the transmittance calculator is further configured to update the placement indication value based on a comparison of the at least one reference transmittance value to the at least one updated operating transmittance value.

33. The noninvasive medical monitoring device of claim 31, wherein the transmittance calculator is further configured to update the placement indication value based on a determined difference between the at least one reference transmittance value and the at least one updated operating transmittance value.

34. The noninvasive medical monitoring device of claim 31, wherein the instructions comprise a direction relative to the reference position in which to move the physiological sensor.

35. The noninvasive medical monitoring device of claim 31, wherein the instructions comprise a visual indication on a display.

36. The noninvasive medical monitoring device of claim 31, wherein the instructions comprise an audible indication output via a speaker.

37. The noninvasive medical monitoring device of claim 31, wherein the monitoring device further comprises a data storage device configured to store the at least one reference transmittance value.

38. The noninvasive medical monitoring device of claim 31, wherein the transmittance calculator is further configured to determine the placement indication value based on a comparison of the at least one reference transmittance value to the at least one operating transmittance value.

39. The noninvasive medical monitoring device of claim 31, wherein the transmittance calculator is further configured to determine the placement indication value based on a determined difference between the at least one reference transmittance value and the at least one operating transmittance value.

40. The noninvasive medical monitoring device of claim 31, wherein the transmittance calculator is further configured to delay determination of a physiological measurement associated with the one or more physiological signals when the placement indication value exceeds the placement indication threshold.
